(19) United States
(12) Patent Application Publication
(10) Pub. No.: US 2026/0007487 A1
(43) Pub. Date: Jan. 8, 2026

(54) CONTROL FACILITY FOR MOBILE MEDICAL DEVICES, AND MOBILE MEDICAL DEVICE

(71) Applicant: Siemens Healthineers AG, Forchheim (DE)

(72) Inventors: Wolfgang ENGE, Bayreuth (DE); Matthias MUELLER, Bamberg (DE); Alexander BEER, Kulmbach (DE); Sebastian MAISENBACHER, Muenchen (DE)

(73) Assignee: Siemens Healthineers AG, Forchheim (DE)

(21) Appl. No.: 19/259,528

(22) Filed: Jul. 3, 2025

(30) Foreign Application Priority Data
Jul. 5, 2024 (DE) 10 2024 206 342.8

(51) Int. Cl. B60K 23/04; A61B 90/50

(52) U.S. Cl. CPC B60K 23/04; A61B 90/50; B60Y 2300/1815

(57) ABSTRACT

A control device for a mobile medical device, comprises a controller and a selection unit. The controller is configured to control and/or regulate a drive unit of the mobile medical device. The mobile medical device includes an operator control device for actuation by a user. The control device is provided with actuation data of the operator control device. The controller has at least two different controller settings, wherein the selection unit is configured to select, set and/or use the controller setting based on the actuation data.

### CROSS-REFERENCE TO RELATED APPLICATION(S)

The present application claims priority under 35 U.S.C. § 119 to German Patent Application No. 10 2024 206 342.8, filed Jul. 5, 2024, the entire contents of which is incorporated herein by reference.

### BACKGROUND

In the medical sector, in particular in hospitals and care facilities, mobile medical devices, for example sickbeds, patient couches, imaging equipment and treatment facilities, are becoming increasingly widely established. Often, these medical devices are heavy appliances each having a mass of 100 kg or more. To make them easier to handle, mobile medical devices are often equipped with a drive, for example a motor, for assistance and/or to allow autonomous and/or automated movement.

In the case of vehicles with their own independent drive, a PI controller is used in order to correct the position or speed of the different axles to fit the predefined setpoint in each case. A PI controller consists of a proportional part and an integral part. The proportional part (described by the control parameter Kp) responds to the current error, that is, to the deviation of the current actual value from the desired setpoint. However, the disadvantage is that the closer the setpoint and feedback get to each other, the correction also becomes smaller and smaller. In theory, the setpoint is never reached. The integral part collects the errors over time and enables a finer tuning of the feedback control system. The I component is able to solve the problem of not reaching the setpoint from the P component. The I component (described by the control parameter Ki) integrates the difference between setpoint and feedback. Even with small differences, after some time the correction signal becomes large enough to correct the error between PID setpoint and PID feedback.

In the environment in which such mobile medical devices are employed, it is necessary to observe high safety standards in order to ensure the safety and wellbeing of operators and/or patients, but also to protect material assets. The handling of such heavy mobile devices is often described by operators as unfamiliar. In particular, the operators find the response behavior to control inputs and the response of the mobile medical device especially noticeable. For this reason a controller is often used in mobile medical devices equipped with a drive unit, the controller forming a feedback control system in conjunction with the input unit and the drive unit.

Vehicles with their own independent drive can be controlled autonomously (vehicle moves toward its destination independently, without human intervention), automatically (specified workflows are performed independently by the vehicle or the software in accordance with specific parameters) or manually. In manual operation, the user controls the trajectory of the vehicle by way of control elements such as steering wheel, force sensor or joystick and thus becomes part of the feedback control system. In this case practice shows that the control of the vehicle is perceived as more agreeable when the direction specification is relayed instantaneously to the actuators by the feedback control system.

A vehicle equipped with a differential drive consists in its simplest form of two driven wheels on an axle together with one or at least two passive, co-rotating jockey wheels which are able to rotate freely. The two driven wheels are mounted on the same axle and are controlled and driven separately. The vehicle is controlled without steered wheels. To negotiate a bend, the drive wheels are driven at different speeds. The delay between direction specification and completion of the desired trajectory is therefore linked directly to the realization of the desired target speeds with respect to time.

The PI controller is particularly suitable for the precise autonomous/automatic operation of the vehicle. In the case of manual control of the vehicle, the PI controller with a small I component or the pure P controller is perceived as particularly pleasant because the output signal is immediately adjusted proportionally to the new setpoint to suit the actuators of the vehicle. The user already attempts personally to correct the constant control deviation of the P controller, and the integration with respect to time of the difference between setpoint and feedback counteracts this once again with a delay. The I component is therefore perceived by the user during manual operation as disruptive and “bothersome”.

Differential drives, which are required to switch over frequently between autonomous/automatic and manual operation, employ a PI controller with control parameters Kp and Ki, which result from a compromise for both operating modes.

### SUMMARY

Independent of the grammatical term usage, individuals with male, female or other gender identities are included within the term.

The subject matter of one or more example embodiments of the present invention is a control facility (also referred to as a control device) for a mobile medical device. In this case the control facility can be embodied in the form of hardware, for example as an electronics module, or as software. The control facility is in particular designed for controlling and/or regulating a drive, in particular a speed, an acceleration and/or a movement direction of the mobile medical device. The mobile medical device may be a sickbed, a patient couch, a mobile head scanner, a mobile imaging device or a mobile treatment unit. The mobile medical device has in particular a mass of at least 100 kg, in particular of at least 500 kg.

The mobile medical device is in particular a drivable medical device, in particular a manually, automatically and/or autonomously drivable medical device. The mobile medical device has in particular a drive unit.

The control facility is connected in a wired or wireless arrangement to the drive unit, in particular to a controller of the control facility, for data purposes. The control facility can be embodied to drive and/or control several wheels simultaneously and/or uniformly via the drive unit. Alternatively, the control facility can be designed to control and/or drive wheels and/or axles separately and/or differently.

The control facility comprises at least one controller. The controller can be embodied as software or as a hardware module. The controller is in particular part of a feedback control system. The feedback control system comprises for example the drive unit and an operator control facility of the mobile medical device. The controller is in particular designed to regulate and/or to control a controlled variable, the controlled variable forming and/or comprising for example a speed, a rotational speed of a wheel, an acceleration, a force, a steering angle and/or some other drive variable. The controller is preferably implemented as a continuous controller. The controller comprises and/or can be described by parameters, in particular a set of parameters. The parameter set can for example comprise a proportionality factor and an integration constant for describing a PI controller.

The control facility comprises a selection unit. The selection unit can be realized in the form of software or hardware. The selection unit is preferably connected for data purposes to an operator control facility of the mobile medical device and/or to the controller. The selection unit is designed in particular to select and/or decide between options based on an input variable. In particular, the selection unit is designed to select and/or set a behavior and/or a setting of the controller based on actuation data that is provided by the operator control facility.

Mobile medical devices with their own independent drive can be controlled autonomously (vehicle moves toward its destination independently, without human intervention), automatically (specified workflows are performed independently by the vehicle or the software in accordance with specific parameters) or manually. In manual operation, the user controls the trajectory of the vehicle by way of control elements such as steering wheel, force sensor or joystick and thus becomes part of the feedback control system.

The drive unit may in particular comprise one or more motors, for example in order to drive and/or control an axle and/or wheels of the mobile medical device. The drive unit can in particular be part of a chassis and/or a running gear of the medical device. The drive unit is preferably connected to the controller for data purposes.

The mobile medical device comprises an operator control facility (also referred to as an operator control device). The operator control facility comprises for example an input device (or input means), a switch, a contact blade, a force sensor, a joystick or a pushbutton. The operator control facility is designed to be operated and/or actuated by a user. The user can use the operator control facility to signal to the control facility by way of actuation data that the user is in contact with the mobile medical device and/or would like to take over active control. For example, the operator control facility may comprise and/or form a deadman unit which is designed to signal the presence of an active user via actuation data. In other words, the actuation data may comprise information indicating whether a user is using the mobile medical device, wishes to control and/or regulate it or whether no user is present.

A PI controller is suitable in particular for the precise autonomous/automatic operation of the mobile medical device. In the case of manual control of the mobile medical device, the PI controller with a small I component or the pure P controller is perceived as particularly pleasant because the output signal is immediately adjusted proportionally to the new setpoint to suit the actuators of the vehicle. The user already attempts personally to correct the constant control deviation of the P controller, and the integration with respect to time of the difference between setpoint and feedback counteracts this once again with a delay. The I component is therefore perceived by the user during manual operation as disruptive and “bothersome”.

The control facility, in particular the selection unit, is provided with the actuation data. By being provided with the actuation data, the control facility, in particular the selection unit, is furnished with the information indicating whether a user is present and/or is taking over control or whether no user is present. For example, the actuation data can include the position and movement of the control lever, which indicate whether a user has taken over control.

The controller comprises at least two controller settings. The at least two controller settings are different from one another; in particular these can be reflected in a controller behavior and/or in different parameters. For example, one controller setting could be a PI controller with fast response times, while another controller setting represents a PI controller with slow response times. The controller can be operated in the at least two different controller settings. In particular it is possible to switch over between the different controller settings. For example, one controller setting describes a PI controller having a first parameter set and another controller setting describes a PI controller having a different parameter set.

The selection unit is designed to select one controller setting of the at least two controller settings based on the actuation data and/or to set or use the selected controller setting for the operation of the controller. This means that the selection unit responds in real time to the changes in the actuation data and selects the optimal controller settings. In other words, the selection unit is designed to switch between the controller settings based on the actuation data. For example, the selection unit is designed to select the controller behavior and/or the controller settings and/or to switch between these based on the knowledge of whether a user is present and/or would actively like to take control. Thus, for example, upon detecting an active user, the selection unit could choose a controller setting with a smaller integration component which allows a more direct control.

One or more example embodiments of the present invention are based on the consideration of switching between different controller settings and/or controller behaviors based on evaluation of the actuation data, instead of using a controller having a controller behavior and/or a controller setting which forms a compromise between several possibilities. This permits optimal controller settings and/or controller behaviors to be used in each case based on the actuation data, for example whether a user is present or not. For example, it is thus possible, in the event of control being taken over by a user, to use a controller setting and/or a controller behavior that is pleasant for the user, for example a controller setting with a small integration component, and, if a user is not present, for example in the event of automatic and/or autonomous control, to choose a controller setting which is not required to take the sensitivities of users into consideration.

In addition, the selection unit can take into account whether the user is in a stressful situation. For example, additional sensors could be used for monitoring the user state, such as heart rate sensors or eye movement trackers, in order to further optimize the controller settings.

Generally, it can be said that the control unit according to one or more example embodiments of the present invention responds in a flexible manner to different situations and user states in order to provide the optimal controller settings at all times. This leads to an improved user experience and a more efficient control of the system.

It is particularly preferred that the at least two controller settings comprise a first controller setting and a second controller setting. The first controller setting comprises a first controller behavior, whereas the second controller setting comprises a second controller behavior. The controller behavior can be described for example via different parameters and/or parameter sets. For example, the controller behavior can describe whether the controller is a P controller, a PI controller or a PID controller. Further, the controller behavior can describe the components of different variables, for example the proportional component and/or the integration component. The proportional component, in particular the control parameter Kp, and the integration component, in particular the control parameter Ki, are preferably part of a parameter set for describing the controller behavior of the controller. The selection unit is in this case designed to select a controller behavior on the basis of the actuation data, in particular to switch over between the controller behaviors. This means that the selection unit can respond in real time to changes in the actuation data in order to apply the appropriate controller behavior in a given case. In particular, the selection unit is designed to select, based on the actuation data, whether the first controller setting having a first controller behavior is applied and/or used by the controller or whether the second controller setting having a second controller behavior is applied and/or used by the controller.

It is particularly preferred that the first controller behavior of the first controller setting describes and/or corresponds to a controller behavior of a PI controller. In particular, the first controller behavior comprises an integration component and/or a control factor Ki not equal to zero. In particular, the first controller behavior can also correspond to a PID controller. The selection unit is preferably designed to select the first controller behavior and/or a controller behavior of a PI controller for actuation data which comprises the presence of an active user and/or the information that the user would like to control and/or regulate the mobile medical device.

Furthermore, it is preferably provided that the second controller behavior of the second controller setting corresponds to a controller setting of a P controller or of a PI controller having an integration component which is smaller than the integration component of the first controller behavior. In other words, it is deemed that the integration component and/or control factor Ki of the second controller setting are/is smaller than that of the first controller behavior.

A further subject matter is formed by a mobile medical device, for example a mobile patient couch, a mobile patient bed, a mobile imaging unit and/or a mobile treatment unit. The mobile medical device is in particular a user-controllable and automatically and/or autonomously operated mobile medical device. The mobile medical device has a chassis, the chassis comprising at least three wheels and/or at least two axles. At the same time it can be provided that driven and non-driven wheels are present. The mobile medical device comprises a drive unit, the drive unit being designed to drive at least one of the wheels and/or at least one of the axles. By driving may be understood in particular both acceleration and braking. For example, it is provided that during active control by a user or during user-based control of the mobile medical device, the wheels and/or axles are actively driven such that the mobile medical device has and/or maintains a speed greater than zero. In this case it is provided that, for example during an automatic and/or autonomous operating mode of the mobile medical device, the drive unit brakes the wheel, the wheels and/or the axles such that the mobile medical device comes to a halt. The drive unit preferably comprises at least one electric motor. The drive unit can be designed to drive axles and/or wheels independently of one another.

The mobile medical device comprises an operator control facility, the operator control facility being embodied as described in the context of the control facility. The mobile medical device further comprises the control facility according to one or more example embodiments of the present invention. The operator control facility is connected to the control facility for data purposes and is designed to provide the actuation data. The selection unit is designed to select, set and/or apply one of the controller settings and/or controller behaviors based on the provided actuation data for the controller. In particular, the selection unit is designed to switch over between the controller settings and/or controller behaviors of the controller based on the actuation data.

It is particularly preferred that the drive unit forms and/or comprises a differential drive. The differential drive is designed to drive at least two wheels, in particular differently and/or at different speeds. For example, the mobile medical device can be steered via the differential drive. The controller is designed to regulate and/or control the differential drive, in particular to regulate and/or control the driving of individual wheels and/or axles, preferably differently and/or independently.

An embodiment of the present invention provides that the control facility is designed to regulate and/or control drive parameters of the drive unit. The drive parameters describe for example how a particular wheel and/or an axle are/is driven. For example, the drive parameters can describe whether the driving action concerns a positive or negative acceleration, a desired speed, an axle or a wheel position. The drive parameters preferably comprise a position and/or speed of an axle and/or a wheel. For example, the drive parameters describe parameters of the feedback control system of the controller.

A particularly preferred embodiment of the present invention provides that the operator control facility comprises at least one input device (or input means) for inputting user control data. The input can for example comprise a joystick, a steering wheel, a slider or a rotary controller, a switch, a contact sensor or a touchscreen. The user control data can for example describe the force of the acceleration of the drive, the desired speed of the mobile medical device, the direction (steering angle) and other information that is necessary for the steering and directed movement of the device by the user.

The controller is preferably configured such that in the first controller setting and/or the first controller behavior it regulates and/or controls the drive unit independently of the user control data. It can further be provided that the controller is configured such that in the first controller setting and/or the first controller behavior it does not evaluate the user control data or does not use it for the feedback control. The first controller setting and/or the first controller behavior in which the user control data does not contribute to the control correspond/corresponds in particular to a PI controller, a PID controller or a controller having an integration component not equal to zero. This is possible since the integration component may be perceived as unpleasant during control by the user, whereas in a feedback control system controlled independently of the user it has no negative effects.

In this embodiment, it is further preferably provided that in the second controller setting the user control data serves as a basis for regulating and/or controlling the drive unit. In the second controller setting, the user control data is in particular evaluated and/or used in the controller and affects the actuation, control and regulation of the drive unit. It is provided in particular in this case that in the second controller setting, in which the user control data is fed into the feedback control system, a controller behavior of a PI controller or of a P controller having a small integration component or without an integration component is used. By using a controller behavior having a small I component or without an I component, the user perceives the control behavior of the controller as particularly pleasant in the second controller setting. This embodiment enables the mobile medical device to be optimally controlled depending on the use of the user control data.

It is particularly preferred that the user control data comprises an input speed, an input direction, an input position, an input steering and/or an input acceleration.

It is also particularly preferred that the operator control facility comprises a deadman unit. The deadman unit can for example comprise a deadman's switch or a sensor which describes the contact with an active user. The deadman unit is designed to provide deadman data that describes whether an actuation by an active user is present, for example whether a user wishes to control, regulate and/or operate the mobile medical device, or whether no such active user is present. If, for example, no active user is present, it is provided that the mobile medical device is actively braked so as not to cause any safety risks for patients, operators, persons or objects in the environment. However, if an active user actuation is present, the drive unit is intended to follow the inputs of the user. The actuation data comprises the deadman data. The control facility is designed to select or apply a controller setting for user-based control if an active user actuation is present. However, if no active user actuation is present, it is provided that the control facility selects and/or sets a controller setting for automatic control.

Preferably, the controller setting for user-based control corresponds to the second controller setting and/or to a PI controller or a PI controller with a small I component.

It is further preferably provided that the controller setting for automatic control corresponds to the first controller setting or to the behavior of a PI controller.

Particularly preferably, if user-based control is present, the controller is designed to use the user control data as a basis for regulating and/or controlling the drive unit.

It is particularly preferred that the mobile medical device is formed by a mobile imaging device such as a mobile C-arm or a mobile head scanner. These medical devices have a high net weight, in which case the behavior of the control facility according to one or more example embodiments of the present invention leads to a particularly pleasant and safe mobility of the medical device.

A further subject matter of one or more example embodiments of the present invention is formed by a method for regulating and/or controlling a drive unit of a real medical device. An operator control facility of the mobile medical device provides the selection unit of the control facility with actuation data. Based on the actuation data, the selection unit selects a controller setting for the controller of the control facility and sets and/or uses the selected controller setting.

### BRIEF DESCRIPTION OF THE DRAWINGS

Further advantages, effects and embodiments of the present invention will become apparent from the attached figures and their description. In the figures:

FIG. 1 shows an exemplary embodiment of a mobile medical device;

FIG. 2 shows a detail from a feedback control system comprising an exemplary embodiment of the control facility;

FIG. 3 shows a flowchart for illustrating the control facility.

### DETAILED DESCRIPTION

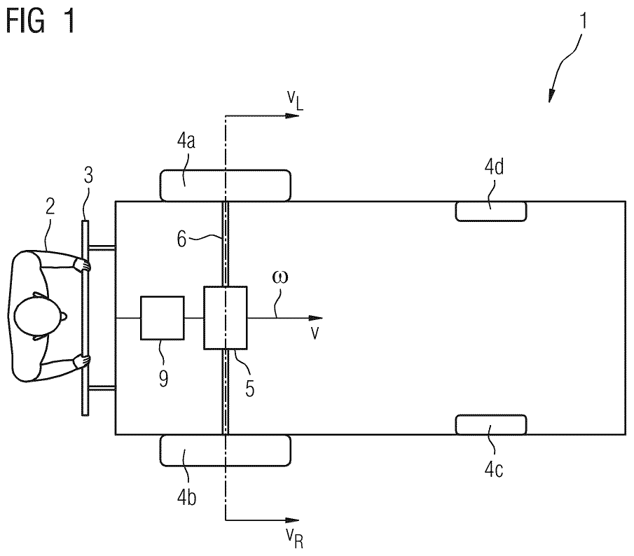

FIG. 1 shows a mobile medical device, which is embodied for example as a mobile head scanner or a mobile C-arm. In this arrangement, the actual imaging unit, for example the C-arm, can be mounted on a drivable structure. The mobile medical device 1 can be controlled, in particular driven and/or steered, by a user 2. For this purpose, the mobile medical device 1 comprises an operator control facility 3. The operator control facility 3 may for example comprise force sensors, which preferably serve as a deadman's switch. The operator control facility 3 may further comprise at least one input device (input means), for example a joystick, pushbuttons or a steering wheel, by which the user 2 is able to control and/or steer the movement, the driving behavior and/or the travel of the mobile medical device 1.

The mobile medical device 1 comprises a chassis which may for example comprise a frame construction and/or be connected to the latter. The chassis and/or the mobile medical device 1 comprise a plurality of wheels 4a to 4d, the wheels 4a and 4b forming driven wheels and the wheels 4c and 4d being co-rotating jockey wheels. The wheels 4a and 4b are connected to a drive unit 5, for example via an axle 6. The drive unit 5 is embodied to drive and/or brake the wheels 4a and 4b. The drive unit 5 is preferably a differential drive so that the wheels 4a and 4b can be driven at different rates in order to enable cornering via different rotational speeds of the wheels 4a and 4b, for example by the angle w. In particular, the user 2 can set and/or choose a desired speed and/or select and/or specify the steering angle ω.

The operator control facility 3 is designed to provide actuation data 7, the actuation data 7 containing for example information about whether the deadman facility has been actuated and/or whether a contact with the user 2 is present or whether the user 2 is no longer in contact with the operator control facility 3 and is no longer actively taking over control. The actuation data 7 may further comprise user control data 8 which includes a desired speed and/or a steering angle.

The mobile medical device 1 comprises a control facility 9, which is arranged in the data stream between the operator control facility 3 and the drive unit 5.

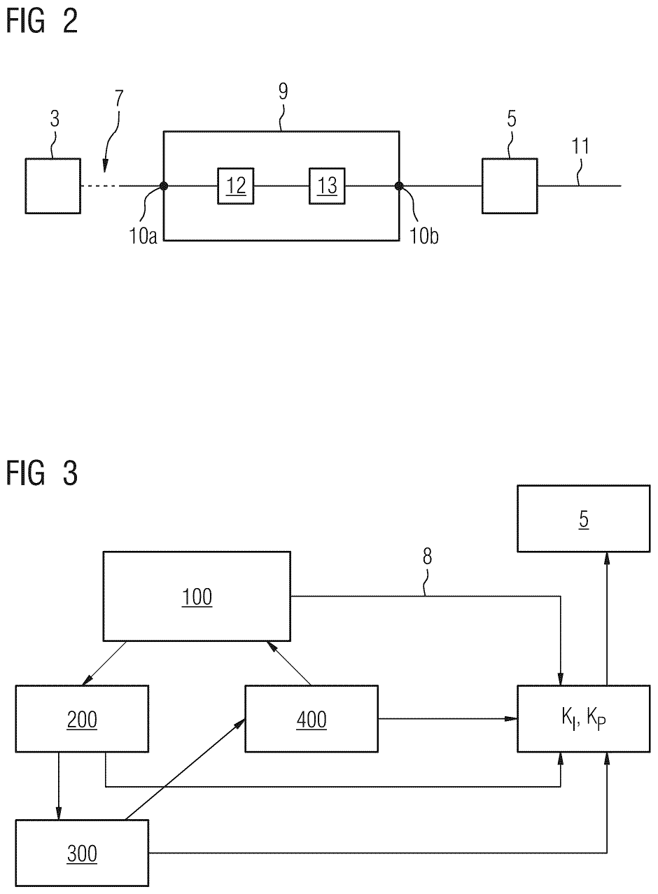

In order to illustrate the processes for regulating and/or controlling the mobile medical device 1 via the control facility 9, FIG. 2 shows a detail from the data and/or signal connections of the control facility 9. The control facility 9 is connected to the operator control facility 3 as well as to the drive unit 5 for data purposes. To that end, the control facility 9 has a data input 10a and a data output 10b. The control facility 9 is provided with actuation data 7 by the operator control facility 3 at the data input 10a. In this case the actuation data 7 can comprise the user control data 8 and/or deadman data. The control facility 9 provides controller signals and/or control signals for regulating and/or controlling the drive unit 5 at the data output 10b. The drive unit 5 responds to the provided signals by adjusting the drive parameters 11, for example by adjusting the rotational speed of the wheels 4a and 4b.

The control facility 9 comprises a selection unit 12 and a controller 13. The selection unit 12 is preferably connected to the data input 10a. In particular, the selection unit 12 is arranged between the data input 10a and the controller 13. The selection unit 12 is provided with the actuation data 7. Based on the actuation data 7, the selection unit 12 selects a controller setting to be used and/or to be set for the controller 13 from at least two possible controller settings. In particular, the selection unit 12 selects the optimal and/or most suitable controller settings for the currently available actuation data 7 and communicates these to the controller 13. For this purpose, the selection unit 12 can for example provide and/or communicate a parameter set, preferably comprising KI and KP, and/or the controller setting to the controller 13. The controller 13 then uses the selected controller setting for regulating and/or controlling the drive unit 5. In active control by the user 2, the controller 13 also uses the user control data 8 in addition to the controller setting, whereas in an automatic control mode, i.e. control without the user 2, the user control data 8 is not used by the controller for regulating or controlling the drive unit 5.

To illustrate this, FIG. 3 schematically shows a workflow of the processes and/or operations in the control facility 9. In step 100, the operator control facility 3 is operated by the user and/or the deadman data confirms that an active user is present. In addition, the actuation data 7 comprises user control data 8 that describes the form of control desired by the user 2. In this state, in which the deadman unit is operated by the user, the selection unit 12 initiates the use of the second controller setting, which provides a small integration component so that the controller can respond with a small time delay to the user control data 8. In this case the controller actuates the drive unit 5 based on said data.

In step 200, it is detected that the deadman unit has been released by the user 2, thus necessitating a switchover from user-based control to an automatic control mode. For this purpose, the selection unit 12 now causes 300 the controller 13 to use the first controller setting, which has a larger integration component. After the controller 13 has been prompted to use the first controller setting, the controller 13 no longer uses the user control data 8, but instead uses braking control data, which for example provides a maximally permissible braking of the mobile medical device 1. This providing of the data can be accomplished via the selection unit 12 and/or already be provided in the controller 13.

In step 400, it is detected that the user 2 is again actuating the deadman unit. The selection unit 12 now causes the controller once again to use the second controller setting and the feedback control system and/or control are/is once again in state and/or step 100, in which the user control data 8 is once again used for control and/or regulation.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A control device for a mobile medical device, the control device comprising:
a controller configured to at least one of control or regulate a drive unit of the mobile medical device, wherein
the mobile medical device includes an operator control device for actuation by a user,
the control device is provided with actuation data of the operator control device, and
the controller has at least two different controller settings; and
a selection unit configured to at least one of select, set or use a controller setting, among the at least two different controller settings, based on the actuation data.

2. The control device as claimed in claim 1, wherein
the at least two different controller settings include a first controller setting and a second controller setting, the first controller setting having a first controller behavior and the second controller setting having a second controller behavior, and
the selection unit is configured to at least one of select, set or use the first controller setting or the second controller setting based on the actuation data.

3. The control device as claimed in claim 2, wherein the first controller behavior of the first controller setting corresponds to a PI controller.

4. The control device as claimed in claim 2, wherein the second controller behavior of the second controller setting corresponds to a P controller or to a PI controller with a smaller I component than a PI controller of the first controller setting.

5. A mobile medical device comprising:
a chassis with wheels;
a drive unit configured to drive at least one of the wheels;
the control device as claimed in claim 1; and
the operator control device, wherein the operator control device is configured to provide the control device with the actuation data.

6. The mobile medical device as claimed in claim 5, wherein the drive unit forms a differential drive for driving at least two of the wheels.

7. The mobile medical device as claimed in claim 5, wherein
the control device is configured to at least one of regulate or control drive parameters of the drive unit, and
the drive parameters include at least one of a position or a speed of at least one of an axle or a wheel.

8. The mobile medical device as claimed in claim 5, wherein
the at least two different controller settings include a first controller setting and a second controller setting, the first controller setting having a first controller behavior and the second controller setting having a second controller behavior, and
the selection unit is configured to at least one of select, set or use the first controller setting or the second controller setting based on the actuation data.

9. The mobile medical device as claimed in claim 8, wherein the operator control device comprises:
at least one input device to input user control data, and wherein at least one of
the controller is configured to at least one of regulate or control the drive unit independently of the user control data in the first controller setting, or
the controller is configured to use the user control data as a basis for at least one of regulating or controlling the drive unit in the second controller setting.

10. The mobile medical device as claimed in claim 9, wherein the user control data includes at least one of an input speed, an input direction or an input position.

11. The mobile medical device as claimed in claim 8, wherein the operator control device comprises:
a deadman unit configured to provide deadman data, wherein the deadman data describes whether an active user
actuation is present or not,
the actuation data includes the deadman data, and
the control device is configured to at least one of at least one of select, set or use a controller
setting for user-based control when an active user
actuation is present, or
at least one of select, set or use a controller setting for automatic control when an active user actuation is not present.

12. The mobile medical device as claimed in claim 11, wherein the controller setting for user-based control corresponds to at least one of a P controller or to a PI controller with a smaller I component than the first controller setting.

13. The mobile medical device as claimed in claim 11, wherein the controller setting for automatic control corresponds to at least one of the first controller setting or a PI controller.

14. The mobile medical device as claimed in claim 11, wherein at least one of
when user-based control is present, the controller is configured to use user control data as a basis for at least one of regulating or controlling the drive unit, or
when an automatic control mode is present, the controller is configured to actuate the drive unit to brake the mobile medical device.

15. The mobile medical device as claimed in claim 5, wherein the mobile medical device includes at least one of a mobile head scanner, a mobile C-arm, a mobile patient couch or a mobile imaging device.

16. The control device as claimed in claim 3, wherein the second controller behavior of the second controller setting corresponds to a P controller or to a PI controller with a smaller I component than the PI controller of the first controller setting.

17. The mobile medical device as claimed in claim 6, wherein
the control device is configured to at least one of regulate or control drive parameters of the drive unit, and
the drive parameters include at least one of a position or a speed of at least one of an axle or a wheel.

18. The mobile medical device as claimed in claim 5, wherein the operator control device comprises:
a deadman unit configured to provide deadman data, wherein the deadman data describes whether an active user
actuation is present or not,
the actuation data includes the deadman data, and
the control device is configured to at least one of at least one of select, set or use a controller
setting for user-based control when an active user
actuation is present, or
at least one of select, set or use a controller setting for automatic control when an active user actuation is not present.

19. The mobile medical device as claimed in claim 7, wherein
the at least two different controller settings include a first controller setting and a second controller setting, the first controller setting having a first controller behavior and the second controller setting having a second controller behavior, and
the selection unit is configured to at least one of select, set or use the first controller setting or the second controller setting based on the actuation data.

20. The mobile medical device as claimed in claim 19, wherein the operator control device comprises:
a deadman unit configured to provide deadman data, wherein the deadman data describes whether an active user
actuation is present or not,
the actuation data includes the deadman data, and
the control device is configured to at least one of
at least one of select, set or use a controller setting for user-based control when an active user actuation is present, or
at least one of select, set or use a controller setting for automatic control when an active user actuation is not present.
